(19) United States
(12) Patent Application Publication
(10) Pub. No.: US 2003/0199943 A1
(43) Pub. Date: Oct. 23, 2003

(54) Method and device for maintaining wakefulness and preventing undesired sleep

(76) Inventors: Orly Katz, Weston, CT (US); Amiram Katz, Weston, CT (US)

Correspondence Address: Paul A. Fattibene, Fattibene and Fattibene, 2480 Post Road, Southport, CT, 06490, US

(21) Appl. No.: 10/124,048

(22) Filed: Apr. 17, 2002

(51) Int. Cl. A61N 1/18

(52) U.S. Cl. 607/48

(57) ABSTRACT

A cuff having stimulating electrodes providing a wakefulness electrical signal and a counter sleepiness electrical signal and a sleepiness detector coupled to a control unit. The sleepiness detector detects an indicator of the onset of sleep, such as the heart rate, and provides a signal to the control unit. The control unit analyzes the heart rate and determines signs of sleepiness. Upon the detection of signs of sleepiness, the counter sleepiness electrical signal is provided to the stimulating electrodes, which contract muscles in a user, preferably the calf muscles. The muscle contraction provides a stimulus and improves circulation that prevents undesired sleep and maintains wakefulness. Alertness is also increased. The present invention is an aid in preventing sleepiness for use in many activities, such as prolonged driving, and performing other tedious or routine tasks. The invention also helps to improve alertness and fend off the onset of sleepiness increasing productivity and avoiding potentially dangerous accidents.

### FIELD OF THE INVENTION

This invention relates in general to an electronic device for maintaining wakefulness or alertness, and more particularly to a device worn by a user for detecting signs of sleep and providing stimulation that prevents falling

### BACKGROUND OF THE INVENTION

There are many activities that are done for long periods of time and are monotonous or are done at hours in which individuals may become tired or sleepy. In many situations, it is not desired to have a reduced alertness due to being tired or sleepy, and rest is not practical or cannot be achieved immediately. Sleepiness may reduce reaction time and alertness, resulting in accidents or mistakes, which would not normally occur. Many individuals have had the problem and occurrence of having to fight off sleepiness or tiredness in order to continue or complete a desired task.

Moderate to severe sleep deprivation is believed to be a problem for many Americans, with 36 million admitting that it affects their daily activities or performance on the job. Many motor vehicle accidents are a result of driver sleepiness. Sleepiness may be a causative factor in one to three percent of all motor vehicle crashes in the United States. Surveys of the prevalence of sleepy behavior in drivers suggests that sleepiness may be a more common cause of highway accidents than is reflected in the estimates. Most of the sleep related accidents involve passenger vehicles.

Long haul truck drivers were found to obtain less sleep than is required for alertness. The greatest vulnerability to sleep, or sleep-like states, is in the late night and early morning. Other factors are known to enhance sleep besides sleep deprivation. These factors include diminished sensory input, such as driving on a dark straight rural road, boring activities, long and monotonous tasks. Additionally, diminished peripheral circulation such as sitting for long periods of time after a large meal, have also resulted in enhanced sleepiness. Providing sensory stimulation and improving peripheral circulation has been found to improve wakefulness as measured by a performance vigilance test and a maintaining wakefulness test in sleep deprived individuals.

One of the easiest measurable physiological changes occurring in the transition to sleep is the slowing of the heart rate. This was discovered many years ago when it was noted that a general slowing of the pulse occurred with the onset of sleep. Numerous modern sleep researchers have later confirmed this.

Several devices have been suggested to prevent undesired sleep. These devices include relatively complicated and difficult to use devices such as the monitoring of eye closure by cameras, a change in head position detected by specially designed helmets, and other devices. However, none of these devices have gained acceptance due to many factors such as cost, inconvenience and/or ineffectiveness. Accordingly, there is a need for a simple, easy to use device and method that can easily and effectively maintain wakefulness and alertness when undesirable sleep approaches. Such a device and method can benefit numerous drivers, pilots, machine operators, navigators, members of the Armed Forces, watchmen, traffic controllers, shift workers and many others in key and important positions in which dangerous and life threatening situations may occur through approaching sleepiness and reduced alertness.

### SUMMARY OF THE INVENTION

The present invention comprises an electronic cuff that may be placed on the leg of a user to provide a base stimulus to maintain wakefulness. A detector or a sensor for detecting the onset of sleepiness may be used and an additional stimulus may be provided when sleepiness is detected. A controller, upon the detection of sleepiness of the user, signals stimulating electrodes resulting in muscle contractions of the user. The muscle contraction improves circulation as well as provides a stimulus to maintain wakefulness. The device may also be programmed to provide a stimulus at predetermined time intervals in a pattern to improve circulation and prevent the onset of sleepiness.

Accordingly, it is an object of the present invention to maintain wakefulness and detect and prevent sleepiness.

It is another object of the present invention to increase alertness during tedious and monotonous activities.

It is an advantage of the present invention that it is relatively easy to use.

It is a further advantage of the present invention that it is relatively simple, safe, and effective.

It is a feature of the present invention that a base stimulus is used to maintain wakefulness.

It is a feature of the present invention that a sleepiness sensor is used.

It is another feature of the present invention that a stimulating electrode provides additional stimulus upon the detection of the onset of sleepiness.

These and other objects, advantages, and features will become readily apparent in view of the following more detailed description.

### BRIEF DESCRIPTION OF THE DRAWINGS

FIG. 1 diagramatically illustrates one use of the present invention.

FIG. 2 schematically illustrates an embodiment of the present invention worn by a user.

FIG. 3A is a plan view of the inside surface of a cuff of an embodiment of the present invention.

FIG. 3B is a plan view of the outside of a cuff of an embodiment of the present invention.

FIG. 3C schematically illustrates a controller unit that detachably connects to the cuff illustrated in FIGS. 3A-3B.

FIG. 4 is a block diagram illustrating the operation of an embodiment of the present invention.

FIG. 5 is a block diagram illustrating the method steps in practicing the present invention.

FIG. 6 is a graph illustrating a wakefulness signal and a counter sleepiness signal.

### DETAILED DESCRIPTION OF THE PREFERRED EMB D

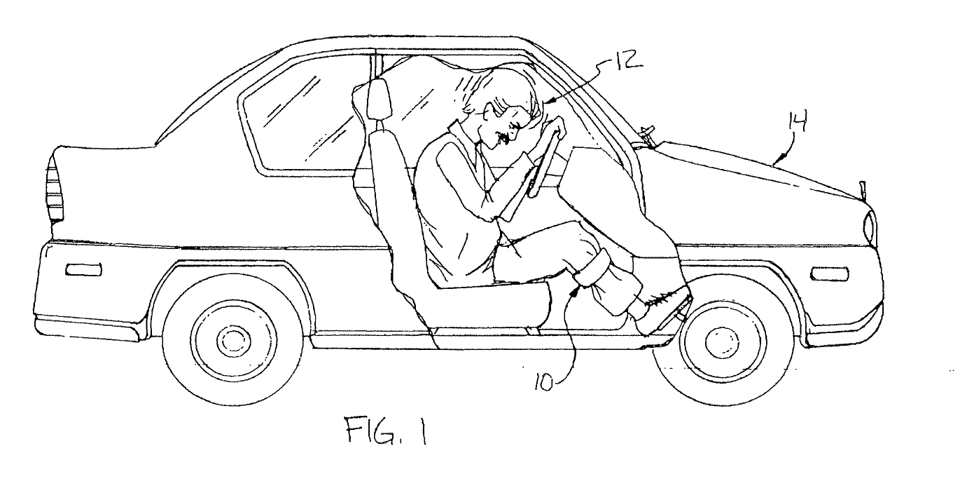

FIG. 1 diagramatically illustrates the application of the present invention to an operator of a motor vehicle. The wakefulness device 10 may be applied to a person or user's 12 leg driving in a car 14. It is common when driving long hours and sitting in a fixed position that undesired sleepiness may occur. The wakefulness device 10 provides a base stimulus to maintain wakefulness and monitors for signs of sleepiness and when detected provides an additional stimulus to improve wakefulness and alertness. A portion of the car 14 has been cut away in the illustration to better illustrate the driver or person 12. The operator of an automobile is only one example of an individual that may benefit from the present invention. It should readily be appreciated that the present invention will benefit numerous individuals that are required to perform tasks or activities while fighting off undesired sleep and in situations where improved alertness or wakefulness is required. These activities may include, but not in any way be limited to, drivers, pilots, machine operators, navigators, members of the Armed Forces, watchmen, traffic controllers, shift workers, and many others in similar activities or roles.

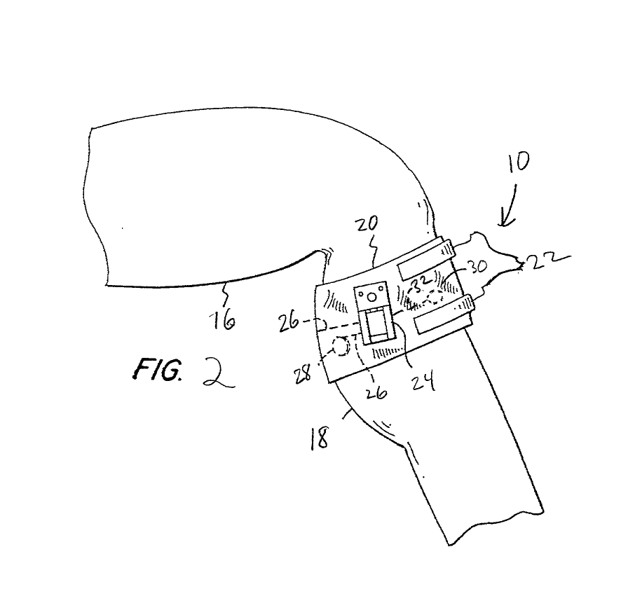

FIG. 2 schematically illustrates the application of an embodiment of the present invention on a user's leg. Placed around the calf 18 of a user's leg 16 is placed the wakefulness device 10. The wakefulness device 10 comprises a cuff 20 with holding means or straps 22 for holding the cuff 20 securely in place on the user's calf 18. Placed on the cuff 20 is a control unit 24. Connected to the control unit 24 by wires 26 is a pair of stimulating electrodes 28. Only one stimulating electrode 28 is visible in FIG. 2, the other stimulating electrode is positioned on the other side of the user's calf 18 and is not visible. Also coupled to the control unit 24 by wire 32 is means for detecting sleepiness or a monitoring electrode 30. The sleepiness or monitoring electrode 30 may be an electrocardiogram (ECG) electrode or other equivalent detector for detecting a change of any physiological indication of the onset of The sleepiness monitoring electrode or detector 30 may detect any physiological indicator of sleepiness. One such physiological indicator of sleepiness is the heart rate. The sleepiness or monitoring electrode or detector 30 may detect the heart rate by use of the electrophysiology or the electrical manifestations of the heart. One of these is the ventricle contraction of the heart and the associated electrical signals provided by volume conduction. A relatively large electrical signal may be detected at nearly any surface location of the body as a result of the relatively large electrical signal associated with the ventricular contraction of the heart. The sleepiness or monitoring electrode 30 easily detects this relatively large electrical signal. Electrocardiogram or ECG signals, or other electrophysiological signals of the heart are easily detected.

Various electrical signals are associated with or generated by the different functions of the heart and are referred to by the letters P, Q, R, S, and T. The R signal is representative of the ventricular contraction. This ventricular contraction can be used to measure the heart rate. Accordingly, intervals between the peaks of the R signal waves in an ECG signal will determine the heart rate. The sleepiness or monitoring electrode or detector 30 can detect sleepiness by detecting a change in the R-R interval of the ECG signal. The controller 24 may use this R-R interval signal to detect a slowdown in the heart rate and, therefore, provide an indication of the onset of sleepiness. While this is one physiological parameter that may be detected to determine sleepiness, other equivalent well known parameters may be detected to determine the onset of sleepiness.

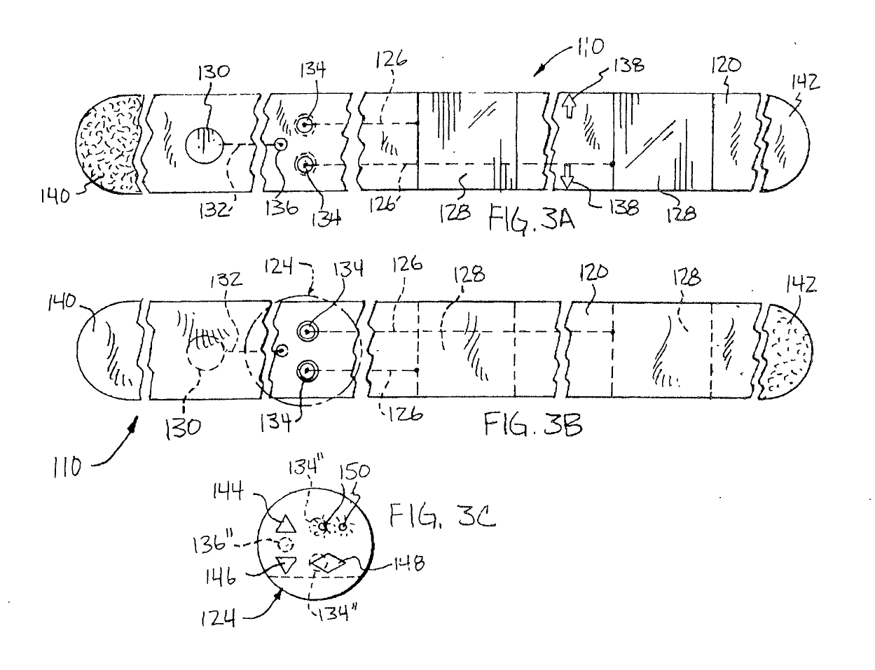

FIGS. 3A-3C illustrate another embodiment of the present invention. FIG. 3A illustrates the inside of a cuff 120 of another embodiment of the wakefulness device 110. The cuff 120 has a pair of stimulating electrodes 128 placed thereon. The stimulating electrodes 128 may be separated anywhere from approximately 2.5 inches or 6.35 cm to approximately 6.5 inches or 16.51 cm. The electrodes 128 may measure approximately 1.75 inches or 4.45 cm on a side. centrally positioned between the stimulating electrodes 128 are locating indicia or arrows 138. The locating indicia 138 help to locate or properly position the stimulating electrodes 128 on the calf of a user. The locating indicia 138 may be positioned at the mid-calf area.

Wires 126 connect the stimulating electrodes 128 to stimulating electrode terminals 134. Also placed on the cuff 120 is a sleepiness monitoring electrode or detector 130. The sleepiness monitoring electrode or detector 130 is connected by wire 132 to a detector terminal 136. On one end of the cuff 120 is holding means or an adhesive portion 140. The adhesive portion 140 is adapted to adhere to the other end 142 of the cuff 120 so that the cuff 120 can encircle a user's calf or leg and be held firmly in position. The distance between the stimulating electrodes 128 and the stimulating electrode terminals 134 may be approximately 4 inches or 10.16 cm, and the distance between the stimulating electrode terminals 134 and the adhesive portion 140 may be approximately 7 to 8 inches or 17.78 to 20.32 cm. The overall length of the cuff may be approximately 23 inches or 58.42 cm.

FIG. 3B illustrates the exterior, outside, or exposed surface of the wakefulness device 110. Placed on this exterior surface of the wakefulness device 110 in the cuff 120 are stimulating electrode connectors 134′ and a monitoring electrode connector 136′. Connectors 134′ and 136′ are adapted to detachably connect to a control unit 124, illustrated in phantom. The connectors 134′ and 136′ may be any type of connector, including a snap connector or equivalent that are well known and used in many electrical FIG. 3C illustrates a control unit 124. The control unit 124 has mating connectors 134″ adapted to connect to the stimulating electrode connectors 134′ on the cuff 120. Additionally, the control unit 124 has a mating connector 136″ adapted to connect to the monitoring electrode connector 136′ on cuff 120. Switches 144 and 146 on the control unit 124 may control the on and off operation, as well as the intensity provided to the stimulating electrodes 128. Additionally, a button 148 may be used to switch the mode of the device and to adjust various settings. Indicator lights 150 may be used to provide information to the user of the status or mode of the control unit 124 or operation of the wakefulness device 110. A battery 152 may be contained in the control unit 124. The control unit 124 may contain conventional electronics so as to detect signals from the sleepiness monitoring electrode 130 and selectively provide signals to the stimulating electrodes 128. The stimulating electrodes 128 provide a mild electric shock and therefore result in muscle contraction. This causes a sensory stimulation as well as enhancing peripheral circulation, which increases alertness and reduces sleepiness.

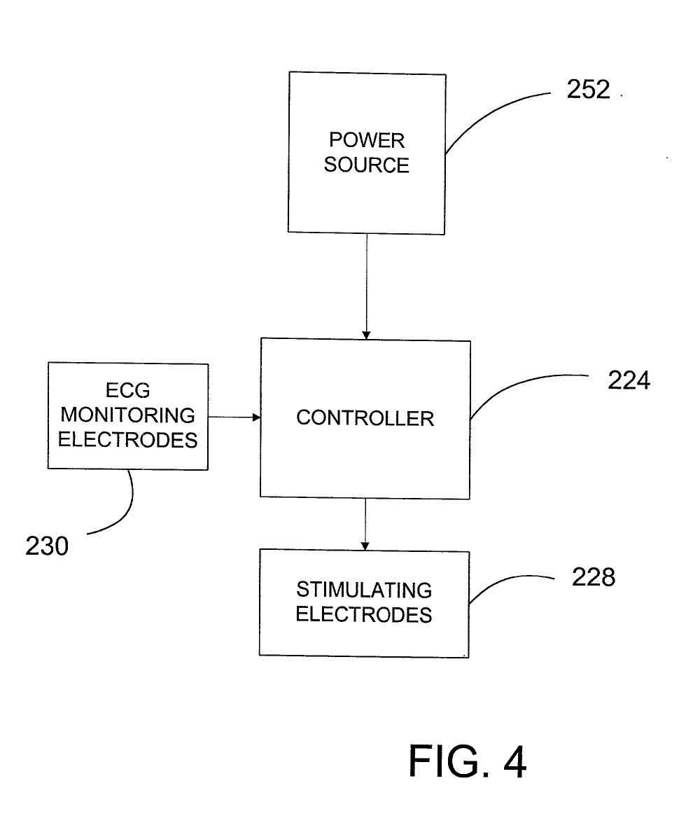

The control unit 124 may be programmed to detect the interval between ventricular contractions, which are an indication of the heart rate. Control unit 124 can analyze the signals and compare the signal to the continuously calculated average of the preceding heart rate or the time between ventricular contractions. When the time between ventricular contractions increases, it is generally an indication of a lower heart rate and the onset of sleepiness. When a predetermined heart rate or rate of decrease of heart rate is detected, the stimulating electrodes 128 may be activated to provide a stimulus or muscle contraction. The muscle contraction will increase peripheral circulation flow and improve alertness. Therefore, wakefulness is improved by providing continuous sensory stimulation, enhancing peripheral circulation, and reacting to drowsiness by creating additional sensory and motor stimulation. Appropriate timing of the activity of the stimulating electrodes 128 may be determined by the rate of increase of the time between the ventricular contractions or other parameters that are indicative of the onset or rate of onset of sleepiness. Additionally, a period may be established for stimulating the electrodes to maintain a target heart rate or degree of alertness or to prevent the onset of sleepiness. FIG. 4 is a block diagram illustrating the operation of the present invention. A power source 252 is coupled to a controller 224. The controller 224 may contain any type of conventional electronics or programmable computer chip to control and analyze signals. Stimulating electrodes 228 are coupled to the controller 224. The stimulating electrodes 228 are placed on a user and provide stimulation for causing a muscle contraction. Also coupled to the controller 224 is a ECG monitoring electrode or sleepiness detector 230. The ECG monitoring electrode monitors and detects the heart rate, which is indicative of sleepiness and provides a signal to the controller 224. The controller 224 analyzes the information to determine or detect the extent of sleepiness of a user. The controller 224 provides a base signal to maintain wakefulness and monitors and analyses signals for signs of sleepiness. Based upon this analysis a signal is provided to the stimulating electrodes 228 providing a stimulus and muscle contraction in the user. The period or rate and strength of the signal are determined by the extent of sleepiness and the individual. The structure and signals provide by the wakefulness detector of the present invention may be similar to the device disclosed in U.S. Pat. No. 6,282,448 entitled “Self Applied and Self Adjusting Device and Method for Prevention of Deep Vein Thrombosis With Movement Detection” issuing to Katz et al on Aug. 28, 2001, which is herein incorporated by reference. For example, a wakefulness or base signal applied to the stimulating electrode for maintaining wakefulness may have a voltage of between 0.1 and 250 volts and a frequency of between 0.05 and 0.5 Hertz with a pulse width between 0.1 and 0.3 milliseconds. It has been discovered that the preferred frequency for the base signal is 0.1 Hertz. Other equivalent wakefulness electrical signals may be used as long as the appropriate stimulus is provided to encourage wakefulness or alertness.

Upon the detection of sleepiness an augmented or additional signal may be provide for providing additional stimulus. The additional signal may have an additional voltage, added to the base voltage of between 5 and 50 volts, that is a total voltage of between 0.1 plus 5 or 5.1 volts and 250 plus 50 or 300 volts, a frequency of between 1 to 20 Hertz for a series of pulses or pulse train having a duration of 0.5 to 3 seconds. It has been discovered that the preferred additional signal to counter sleepiness is an additional voltage of 15 volts, a frequency of 5 Hertz, and a duration of 1 second. Other equivalent counter sleepiness electrical signals may be used as long as the appropriate stimulus is provided to prevent sleepiness.

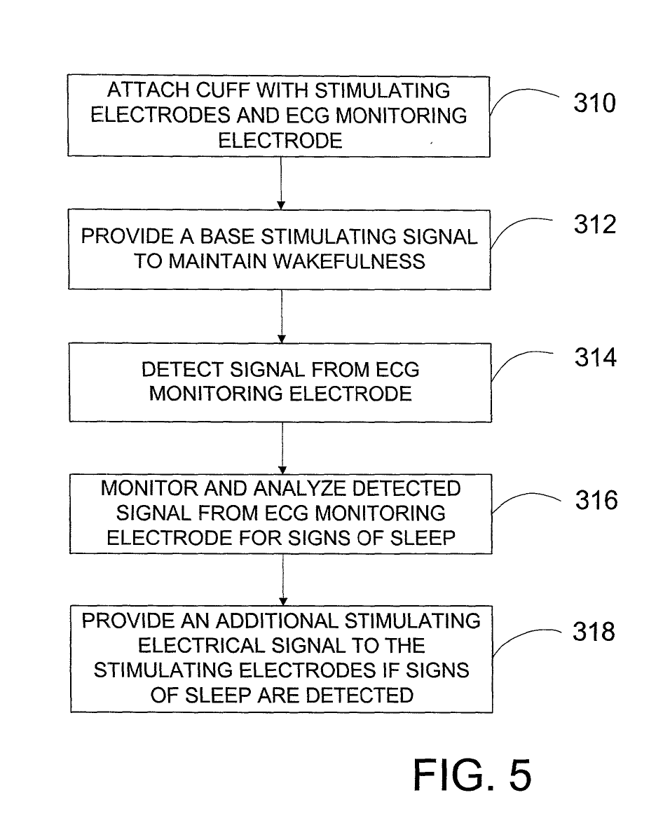

FIG. 5 is a block diagram illustrating the method steps or acts of the present invention. Block 310 represents the method step or act of attaching a cuff with stimulating electrodes and an ECG monitoring electrode or sleepiness detector to a user. Block 312 represents the step of providing a wakefulness or base stimulating signal to maintain wakefulness. Block 314 represents the step or act of detecting a signal from the ECG monitoring electrode or a sleepiness detector. Block 316 represents the method step or act of monitoring and analyzing the detected signal from the ECG electrode or sleepiness detector for signs of sleep. The signs of sleep may be a decreased heart rate, increase time between ventricle contractions of the heart, other equivalent physiological parameters for detecting the onset of sleep or sleepiness. The ECG electrode detects the electrical signals representative of the heart rate. Therefore, the ECG electrode is a type of sleepiness detector. There are other types of sleepiness detectors, which equivalently detect signs of sleepiness. Block 318 represents the method step or act of providing a counter sleepiness or additional electrical signal to the stimulating electrodes if signs of sleep are detected.

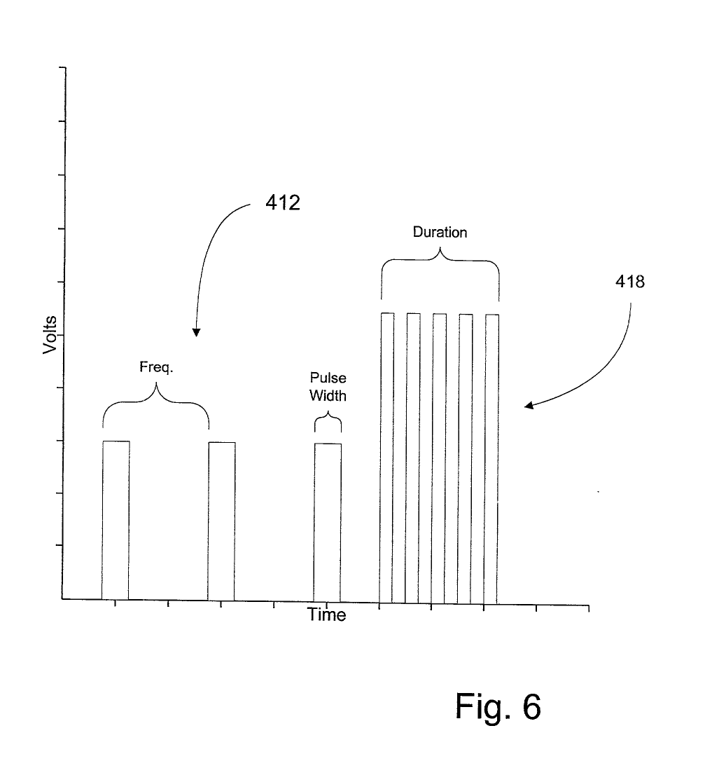

FIG. 6 is a graph illustrating electrical signals provided according to the present invention. In FIG. 6 wakefulness signals 412 are illustrated. The wakefulness signals 412 have a frequency and a pulse width. Counter sleepiness signals 418 are provided in a series of pulses or a pulse train for a predetermined duration upon the detection of sleepiness. A single electrode or a plurality of electrodes may apply the electrical signals to a user.

The present invention provides a relatively simple device that easily detects the onset of sleepiness and provides an electrical stimulation. The electrical stimulation causes contraction of muscles to increase circulation and improve alertness. In preliminary testing of the invention, the maintaining of wakefulness and the prevention of undesired sleep has been substantially increased. Additionally, performance tests of individuals who have been deprived of sleep have improved with when using the present invention.

In one embodiment of the present invention, it may be used as a wakefulness device by providing a wakefulness or base electrical signal to maintain alertness. In another embodiment of the present invention a counter sleepiness or additional electrical signal may be provided when sings of sleepiness are detected. A sleepiness detector may also be used.

Therefore, the present invention has many applications and can be used on individuals to prevent potentially life threatening accidents from occurring due to lack of wakefulness or the onset of sleepiness. Additionally, improve alertness can be achieved.

While the present invention has been described with respect to several particular embodiments, variations and modifications may clearly be made without departing from the spirit and scope of this invention.

What is claimed is:

1. A wakefulness device for maintaining wakefulness and preventing undesired sleep of a user comprising:
a cuff;
a stimulating electrode held by said cuff; and
a control unit coupled to said stimulating electrode, said control unit providing a wakefulness electrical signal to said stimulating electrode, whereby wakefulness is maintained.

2. A wakefulness device as in claim 1 wherein:
said control unit comprises means for analyzing a physiological indicator of sleep.

3. A wakefulness device as in claim 1 further comprising:
a sleepiness detector.

4. A wakefulness device as in claim 3 wherein:
said sleepiness detector comprises a heart rate detector.

5. A wakefulness device as in claim 4 wherein:
the heart rate detector measures an electrical signal that is associated with contraction of the user's heart.

6. A wakefulness device as in claim 1 wherein:
the wakefulness electrical signal comprises a voltage between 0.1 and 250 volts, a frequency of between 0.05 and 0.5 Hertz, and a pulse width of between 0.1 and 0.3 milliseconds.

7. A wakefulness device as in claim 2 wherein:
said control unit selectively provides a counter sleepiness electrical signal to said stimulating electrode.

8. A wakefulness device as in claim 7 wherein:
the counter sleepiness electrical signal comprises a voltage between 5.1 and 300 volts, a frequency between 1 and 20 hertz, and pulse train having a duration of between 0.5 and 3 seconds.

9. A wakefulness device as in claim 8 wherein:
the counter sleepiness electrical signal comprises a voltage of between 5 and 50 volts above a voltage of the wakefulness electrical signal.

10. A wakefulness device as in claim 1 further comprising:
means for attaching said cuff to a user's leg.

11. A wakefulness device as in claim 1 wherein:
said means for attaching said cuff comprises adhesive.

12. A wakefulness device as in claim 1 wherein:
said control unit is detachably connected to said cuff.

13. A wakefulness device for use in maintaining wakefulness and preventing undesired sleep comprising:
a cuff adapted to be placed on a leg of a user;
a stimulating electrode held by said cuff and capable of being positioned on the leg of the user, whereby a muscle of the leg of the user is capable of being stimulated causing contraction of the muscle; and
a control unit coupled to said stimulating electrode, said control unit capable of providing an electrical signal to said stimulating electrode,
whereby the user is prevented from falling asleep and alertness is increased.

14. A wakefulness device for use in maintaining wakefulness and preventing undesired sleep as in claim 13 further comprising:
a sleepiness detector.

15. A wakefulness device for use in maintaining wakefulness and preventing undesired sleep as in claim 14 wherein:
said sleepiness detector comprises a heart rate

16. A wakefulness device for use in maintaining wakefulness and preventing undesired sleep as in claim 13 wherein:
said control unit comprises means for analyzing a physiological indicator of sleep.

17. A wakefulness device for use in maintaining wakefulness and preventing undesired sleep as in claim 16 wherein:
the physiological indicator of sleep comprises a change in heart rate.

18. A wakefulness device for use in maintaining wakefulness and preventing undesired sleep as in claim 14 wherein said control unit comprises:
a wakefulness portion generating and providing a wakefulness electrical signal to said stimulating electrode, whereby wakefulness is capable of being maintained; and
a counter sleepiness portion generating and providing a counter sleepiness electrical signal to said stimulating electrode, whereby undesired sleep is capable of being prevented.

19. A wakefulness device for use in maintaining wakefulness and preventing undesired sleep as in claim 18 wherein:
the wakefulness electrical signal comprises a wakefulness voltage between 0.1 and 250 volts, a frequency of between 0.05 and 0.5 Hertz, and a pulse width of between 0.1 and 0.3 milliseconds.

20. A wakefulness device for use in maintaining wakefulness and preventing undesired sleep as in claim 19 wherein:
the counter sleepiness electrical signal comprises a counter sleepiness voltage between 5 and 50 volts above the wakefulness voltage, a frequency of between 0.1 and 0.5 Hertz, and pulse train having a duration of between 0.5 and 3 seconds.

21. A wakefulness device for use in maintaining wakefulness and preventing undesired sleep as in claim 19 wherein:
the counter sleepiness electrical signal comprises a voltage of between 5 and 50 volts above the wakefulness voltage of the wakefulness electrical signal.

22. A wakefulness device for use in maintaining wakefulness and preventing undesired sleep comprising:
a cuff adapted to be placed on a user;
stimulating means, held by said cuff and positioned on the user, for causing contraction of a muscle of the user;
control means, coupled to said stimulating means, for providing a wakefulness electrical signal to said stimulating means,
whereby wakefulness is maintained.

23. A wakefulness device for use in maintaining wakefulness and preventing undesired sleep as in claim 22 further comprising:
detector means, placed on the user and coupled to said control means, for detecting sleepiness of the user,
whereby sleepiness in the user is capable of being detected by said detector means and a stimulation is provided by said stimulating means resulting in the prevention of undesired sleep and increased alertness.

24. A wakefulness device for use in maintaining wakefulness and preventing undesired sleep as in claim 23 wherein:
said control means comprises means for analyzing a physiological indicator of sleep.

25. A wakefulness device for use in maintaining wakefulness and preventing undesired sleep as in claim 24 wherein:
the physiological indicator of sleep comprises a heart rate change.

26. A wakefulness device for use in maintaining wakefulness and preventing undesired sleep as in claim 22 wherein:
the muscle of the user is a calf muscle.

27. A wakefulness device for use in maintaining wakefulness and preventing undesired sleep as in claim 22 further comprising:
counter sleepiness control portion, associated with said control means, for providing a counter sleepiness electrical signal to said stimulating means.

28. A wakefulness device for use in maintaining wakefulness and preventing undesired sleep as in claim 27 wherein:
the wakefulness electrical signal comprises a voltage between 0.1 and 250 volts, a frequency of between 0.05 and 0.5 Hertz, and a pulse width of between 0.1 and 0.3 milliseconds.

29. A wakefulness device for use in maintaining wakefulness and preventing undesired sleep as in claim 28 wherein:
the counter sleepiness electrical signal comprises a voltage of between 5 and 50 volts above the voltage of the wakefulness electrical signal, a frequency of between 1 and 20 hertz, and a pulse train having a duration of between 0.5 and 3 seconds.

30. A wakefulness device for use in maintaining wakefulness and preventing undesired sleep and increasing alertness comprising:
a cuff adapted to be placed and held in position on a leg of a user;
a removable stimulating electrode held by said cuff and positioned on the leg of the user, whereby a muscle of the leg of the user is capable of being stimulated causing contraction of the muscle and increased circulation;
a heart rate detector, said heart rate detector capable of detecting a heart rate of the user; and
a control unit coupled to said stimulating electrode and said heart rate detector, said control unit capable of providing a wakefulness electrical signal comprising a wakefulness voltage of between 0.1 and 250 volts, a frequency of between 0.05 and 0.5 Hertz, a pulse width of between 0.1 and 0.3 milliseconds and a counter sleepiness electrical signal when said heart rate detector detects a reduced heart rate, the counter sleepiness electrical signal comprising a voltage of between 5 and 50 volts above the wakefulness voltage of the wakefulness electrical signal, a frequency of between 1 and 20 hertz, and a wave train having a duration of between 0.5 and 3 seconds to said stimulating electrode,
whereby the user is capable of maintaining wakefulness and is prevented from falling asleep by increasing alertness.

31. A wakefulness device for use in maintaining wakefulness and preventing undesired sleep and increasing alertness as in claim 30 wherein:
said heart rate detector comprises an electrocardiogram monitoring electrode.

32. A method of maintaining wakefulness and preventing undesired sleep comprising the steps of:
applying a wakefulness electrical signal causing muscle contractions in a user,
whereby the muscle contractions cause a stimulus and increases circulation maintaining wakefulness and preventing undesired sleep and improving alertness.

33. A method of maintaining wakefulness and preventing undesired sleep as in claim 32 comprising the further steps of:
monitoring a user for signs of sleepiness; and
applying a counter sleepiness electrical signal causing muscle contractions in a user when said step of monitoring detects signs of sleepiness.

34. A method of maintaining wakefulness and preventing undesired sleep as in claim 33 wherein:
said step of monitoring a user for signs of sleepiness comprises monitoring a heart rate of a user.

35. A method of maintaining wakefulness and preventing undesired sleep comprising the steps of:
applying a wakefulness electrical signal causing muscle contractions in a user;
monitoring the user's heart rate;
analyzing the user's heart rate for a decreased heart rate, whereby signs of sleepiness are capable of being detected; and
applying a counter sleepiness electrical signal to the user causing muscle contractions in the user when said step of analyzing detects the decreased heart rate,
whereby the muscle contractions cause a stimulus and increases circulation maintaining wakefulness and preventing undesired sleep and improving alertness.

36. A method of maintaining wakefulness and preventing undesired sleep as in claim 35 wherein:
the wakefulness electrical signal comprising a wakefulness voltage of between 0.1 and 250 volts and a frequency of between 0.05 and 0.5 Hertz, and the counter sleepiness electrical signal comprising a voltage of between 5 and 50 volts above the wakefulness voltage of the wakefulness electrical signal, a frequency of between 1 and 20 hertz, and a wave train having a duration of between 0.5 and 3 seconds.

37. A method of maintaining wakefulness and preventing undesired sleep comprising the steps of:
applying a sufficient wakefulness electrical signal to a user's leg so as to improve wakefulness; and
applying a sufficient counter sleepiness electrical signal to the user's leg so as to prevent sleepiness.
